(19) United States
(12) Patent Application Publication
(10) Pub. No.: US 2011/0054552 A1
(43) Pub. Date: Mar. 3, 2011

(54) ELECTRODE LAYOUT METHOD OF HEART TREATMENT APPARATUS

(75) Inventors: Shuichi Takayama, Tokyo (JP); Toshiki Terayama, Tokyo (JP); Hiroki Hibino, Tokyo (JP)

(73) Assignee: OLYMPUS CORPORATION, Tokyo (JP)

(21) Appl. No.: 12/550,772

(22) Filed: Aug. 31, 2009

(51) Int. Cl. A61N 1/08

(52) U.S. Cl. 607/4

(57) ABSTRACT

The purpose is to provide an electrode layout method of a heart treatment apparatus, which is capable of improving the heart treatment efficiency by setting electrodes in the heart as well as reducing the invasion into the patient so as to effectively stimulate a site which needs to be stimulated. There is provided an electrode layout method of a heart treatment apparatus comprising: inserting at least two lines of leads which are provided to the heart treatment apparatus and which have electrodes on their distal ends, into a vein communicated to the interior of a right atrium and extending along a cardiac wall; and placing the electrodes provided on the respective leads in the vein located at approximately opposite positions across a heart.

### BACKGROUND OF THE INVENTION

1. Field of the Invention

The present invention relates to an electrode layout method of a heart treatment apparatus.

2. Description of Related Art

Conventionally, there has been known a body implantable heart treatment apparatus which, upon the occurrence of fibrillation, tachycardia, or bradycardia accompanied by a pump malfunction, outputs electrical pulses from electrodes arranged in the heart to thereby stimulate the myocardium so as to effect recovery of its pump function. The myocardium existing in the left ventricular cardiac wall and the myocardium existing in the right ventricular cardiac wall are synchronously stimulated, by which the both ventricles are sufficiently contracted to achieve the normal pump function.

In PCT International Publication No. WO95/08365 Pamphlet, an epicardial electrode is employed as one of the electrodes arranged in the heart. Generally, an open heart surgery is required to set such an epicardial electrode in a body. On the other hand, there is also known a method in which a portion in the vicinity of a collarbone is incised, a lead is transvenously inserted into the right atrium, and an electrode provided on the lead is anchored in a vein extending from the right atrium along a cardiac wall. In the case of this method, the incision site in the body of the patient can be relatively small. U.S. Pat. No. 6,556,873 has disclosed a technique for facilitating the insertion of a lead into a vein extending along a cardiac wall by varying the flexural rigidity of the lead in a position-dependent manner. PCT International Publication No. WO98/42403 Pamphlet has disclosed a technique for placing an electrode at a desired position in a vein by providing a lead with a stent.

### BRIEF SUMMARY OF THE INVENTION

The present invention takes such a situation into consideration with an object of providing an electrode layout method of a heart treatment apparatus, which is capable of improving the heart treatment efficiency by setting electrodes in the heart as well as reducing the invasion into the patient so as to effectively stimulate a site which needs to be stimulated.

In order to achieve the above object, the present invention provides the following solution.

The present invention provides an electrode layout method of a heart treatment apparatus comprising: inserting at least two lines of leads which are provided to the heart treatment apparatus and which have electrodes on their distal ends, into a vein communicated to the interior of the right atrium and extending along a cardiac wall; and placing the electrodes provided on the respective leads in the vein located at approximately opposite positions across the heart.

### BRIEF DESCRIPTION OF THE SEVERAL VIEWS OF THE DRAWINGS

FIG. 1 illustrates an electrode layout method of a heart treatment apparatus according to one embodiment of the present invention.

FIG. 2 is a cross sectional view of the heart in which electrodes are arranged by the electrode layout method of FIG. 1.

FIG. 3 is a cross sectional view of the heart illustrating a modified example of the electrode layout method of the heart treatment apparatus of FIG. 1.

FIG. 4 illustrates another modified example of the electrode layout method of the heart treatment apparatus of FIG. 1, by which three electrodes are arranged in the heart.

FIG. 5 is a cross sectional view of the heart in which electrodes are arranged by the electrode layout method of FIG. 4.

FIG. 6 illustrates yet another modified example of the electrode layout method of the heart treatment apparatus of FIG. 1, by which three electrodes are arranged in the heart.

FIG. 7 illustrates still another modified example of the electrode layout method of the heart treatment apparatus of FIG. 1, by which four electrodes are arranged in the heart.

### DETAILED DESCRIPTION OF THE INVENTION

Hereunder is a description of one embodiment of the present invention, with reference to drawings.

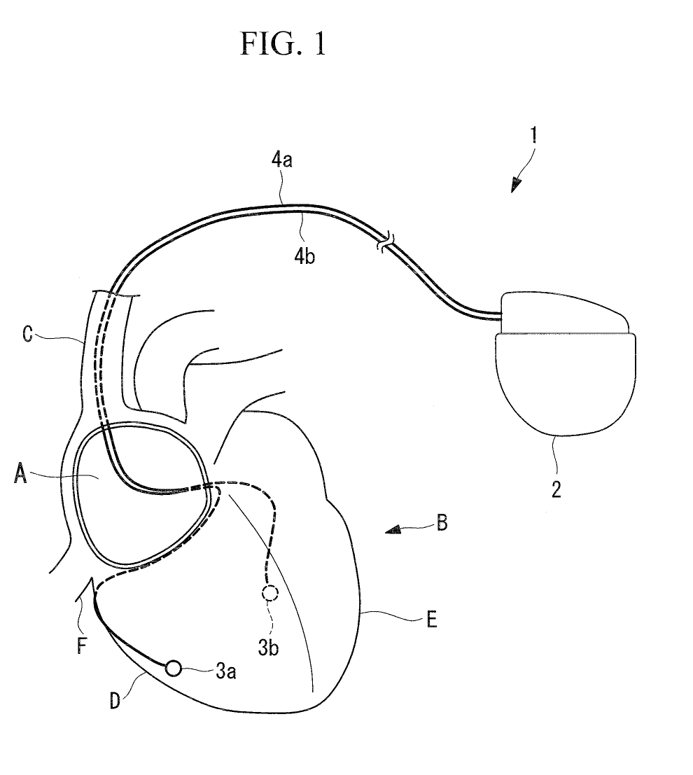

As shown in FIG. 1, in an electrode layout method of a heart treatment apparatus 1 according to this embodiment, two lines of leads 4a and 4b which are connected to an apparatus mainbody 2 and are provided with electrodes 3a and 3b on their distal ends, are inserted in a vein extending from the right atrium A along the cardiac wall, and the respective electrodes 3a and 3b are placed at approximately opposite positions across the heart B. It is needless to say that the electrodes 3a and 3b include well known pacing/sensing electrodes, defibrillation electrodes, or various types of electrodes such as pacing/sensing/defibrillation electrodes.

The heart treatment apparatus 1 according to this embodiment delivers a desired electrical pulse generated in the apparatus mainbody 2 to the electrodes 3a and 3b via the both leads 4a and 4b to thereby apply pacing, defibrillation, or other desired stimuli to the heart B. As for the electrical pulse, there are employed a defibrillation pulse having a relatively large energy for the purpose of defibrillation, and a pacing pulse for regulating the heartbeat. The apparatus mainbody 2 detects the potential change of the electrodes 3a and 3b, by which the heart treatment apparatus 1 acquires an electrocardiographic signal. Based on the acquired electrocardiographic signal, the heart treatment apparatus 1 determines the abnormality of the heartbeat in the heart B, namely, fibrillation, tachycardia, bradycardia, or other heartbeat abnormalities. When the heart treatment apparatus 1 determines the occurrence of fibrillation or tachycardia needing to be treated in the heart B, the heart treatment apparatus 1 delivers defibrillation pulses between the electrodes 3a and 3b. When the heart treatment apparatus 1 determines the occurrence of bradycardia or a heartbeat abnormality needing to be treated in the heart B, the heart treatment apparatus 1 delivers pacing pulses necessary for the treatment to the respective electrodes 3a and 3b.

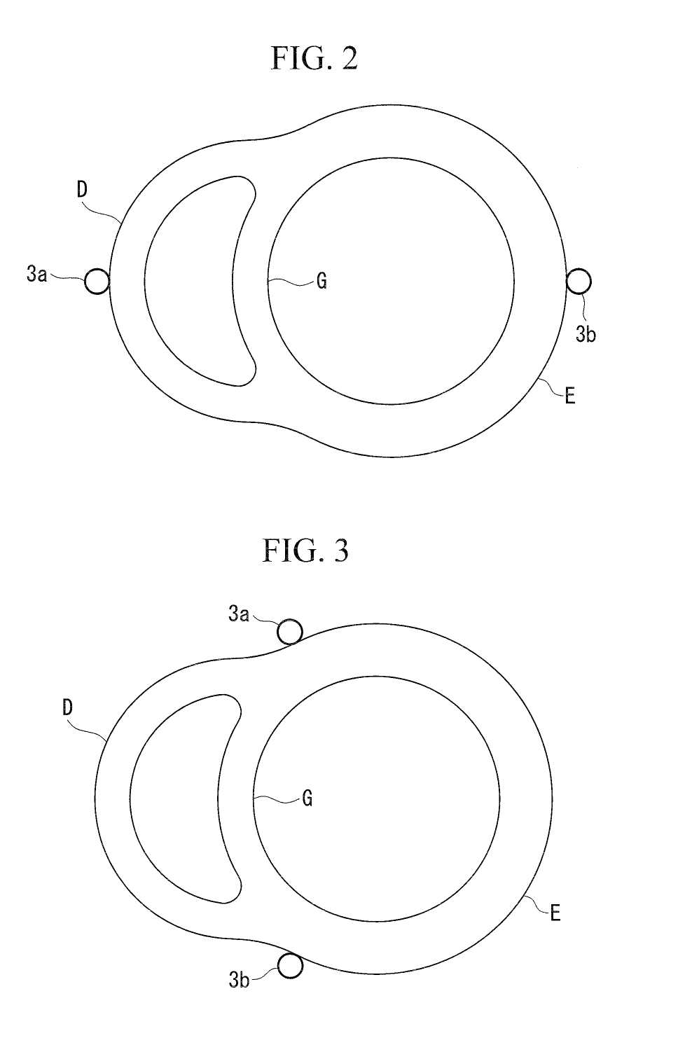

For example, after the vicinity of the collarbone of the patient has been incised, the leads 4a and 4b are inserted from a vein existing near the incision site, through the superior vena cava C, into the right atrium A, and further into the coronary sinus which opens to the interior of the right atrium A. In addition, the lead 4a on one hand is inserted into the small cardiac vein branching from the coronary sinus and extending along the right ventricular free wall D, and the electrode 3a is anchored in the small cardiac vein. The lead 4b on the other hand is inserted into the posterior vein of the left ventricle branching from the coronary sinus and extending along the left ventricular free wall E, and the electrode 3b is anchored in the posterior vein of the left ventricle. As shown in FIG. 2, the thus arranged electrodes 3a and 3b are placed at approximately opposite positions across the whole ventricles.

FIG. 1 illustrates an example where the leads 4a and 4b are inserted from the superior vena cava C into the right atrium A. However, the leads 4a and 4b may also be inserted from the inferior vena cava F into the right atrium A.

Hereunder is a description of the operation of the heart treatment apparatus 1 of which the electrodes 3a and 3b are arranged in such a manner.

Upon the occurrence of fibrillation or tachycardia needing to be treated in the heart B, the heart treatment apparatus 1 according to this embodiment delivers defibrillation pulses between the right ventricular free wall D and the left ventricular free wall E. In addition, upon the occurrence of bradycardia or a heartbeat abnormality needing to be treated in the heart B, the heart treatment apparatus 1 delivers necessary pacing pulses respectively to the right ventricular free wall D and the left ventricular free wall E to regulate the heartbeat.

In this case, according to this embodiment, since the electrodes 3a and 3b are transvenously set in the body, the incision site in the body of the patient can be small as compared to the case of an open heart surgery. By so doing, advantageously, the invasion into the body of the patient can be reduced and physical burdens on the patient caused by the operation can be alleviated.

In addition, by placing the electrodes 3a and 3b at approximately opposite positions across the heart B, and simultaneously delivering pacing pulses to the right ventricular free wall D and the left ventricular free wall E on which the electrodes 3a and 3b are arranged, the left and right ventricles can be synchronously contracted. By so doing, the pump function of the heart B can be effectively recovered, and thereby the treatment efficiency of the heart B can be improved.

Moreover, by placing the electrodes 3a and 3b at approximately opposite positions across the heart B, and delivering defibrillation pulses to the right ventricular free wall D and the left ventricular free wall E which are arranged between the electrodes 3a and 3b, the defibrillation pulses can also be effectively delivered to the ventricular septum G located between the electrodes 3a and 3b. By so doing, advantageously, the ventricular septum G can also be contracted simultaneously with the both ventricular free walls D and E, and thereby the treatment efficiency of the heart B can be further improved.

In the abovementioned embodiment, the lead 4a on one hand is inserted into the small cardiac vein while the lead 4b on the other hand is inserted into the posterior vein of the left ventricle. However, the leads 4a and 4b may also be respectively inserted into other veins communicated to the interior of the right atrium A and existing on the cardiac wall.

Examples of other veins communicated to the interior of the right atrium A can include anterior cardiac veins, the middle cardiac vein and the great cardiac vein. The anterior cardiac veins branch from the right atrium A and extend to the right ventricular free wall D. The middle cardiac vein and the great cardiac vein branch from the coronary sinus and extend to the left ventricular free wall E. Accordingly, even if the lead 4a on one hand is inserted into an anterior cardiac vein and the lead 4b on the other hand is inserted into the middle cardiac vein or the great cardiac vein, the same effect as that of the abovementioned embodiment can be obtained.

In addition, in the abovementioned embodiment, the electrodes 3a and 3b are arranged respectively on the ventricular free walls D and E. However, as shown in FIG. 3, the electrodes 3a and 3b may also be arranged on the cardiac ventricular walls in the vicinities of the boundary between the right ventricle and the left ventricle. This arrangement can also enable effective treatment of the heart B by stimulating the both ventricular free walls D and E as well as the ventricular septum G with electrical pulses delivered between electrodes 3a and 3b.

Moreover, in the abovementioned embodiment, the electrodes 3a and 3b are placed at two positions in the heart B. However, an additional electrode 3c may also be placed at another position differing from the positions of these electrodes 3a and 3b.

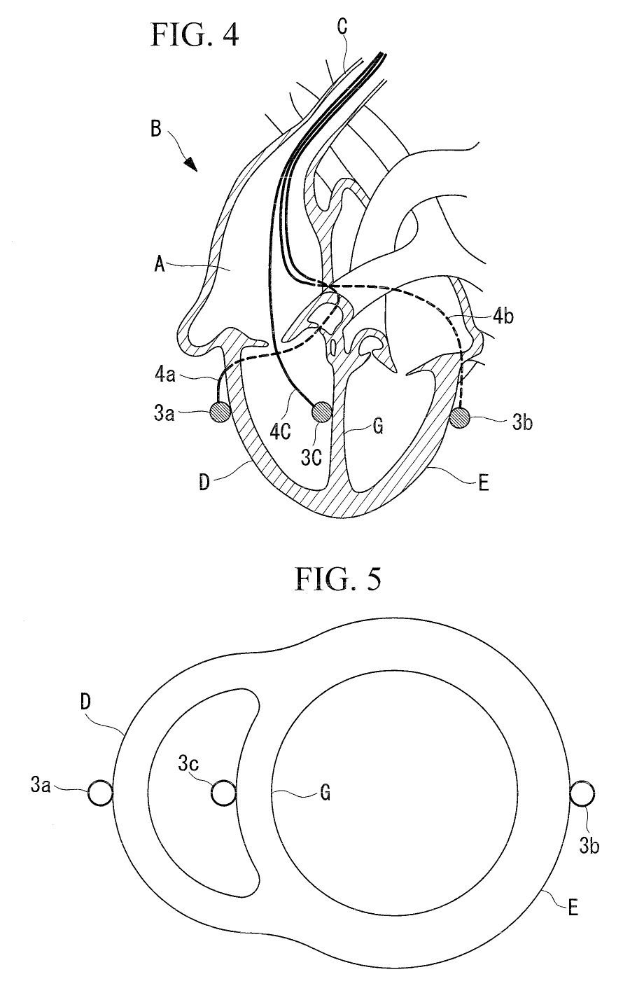

FIG. 4 illustrates an example where an additional lead 4c is inserted from the right atrium A into the right ventricle and the additional electrode 3c is arranged in the right ventricle. By so doing, as shown in FIG. 5, the right ventricular free wall D is arranged between the electrodes 3a and 3c while the left ventricular free wall E is arranged between the electrodes 3b and 3c. By so doing, it becomes possible to selectively stimulate the right ventricular free wall D or the left ventricular free wall E through selection of one or more electrode(s) among the three electrodes 3a to 3c and delivery of electrical pulses thereto.

In this case, the heart treatment apparatus 1 may specify the site where the abnormality occurs and its symptom based on the electrocardiographic signals acquired in the positions of the respective electrode 3a to 3c, and deliver electrical pulses to one or more desired electrode(s) based on the specified site of the occurrence and symptom. By so doing, it becomes possible to selectively deliver necessary electrical pulses to the site needing to be stimulated while suppressing the influence of the electrical pulses to other sites. Alternatively, in a case of defibrillation, the heart treatment apparatus 1 may select two electrodes among the three electrodes 3a to 3c, and deliver defibrillation pulses thereto. In a case where the heartbeat is not recovered to the normal status, the heart treatment apparatus 1 may also select a different combination of two electrodes and again deliver defibrillation pulses thereto.

Furthermore, the additional electrode 3c may also be arranged in a coronary vein, the right atrium A, the superior vena cava C, or the inferior vena cava F.

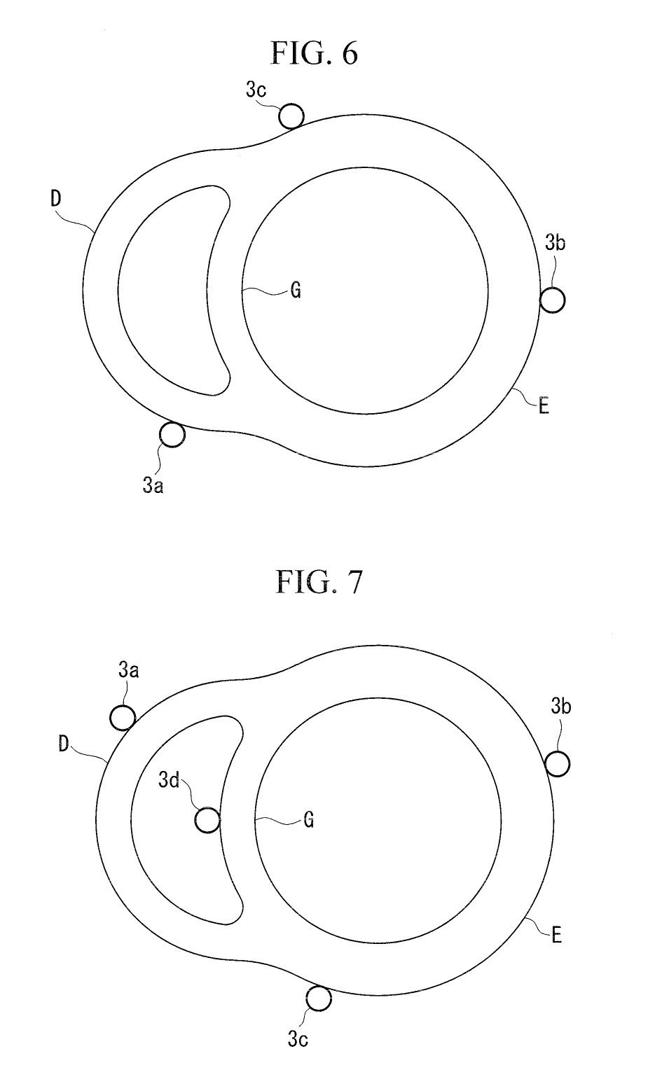

FIG. 6 illustrates an example where the additional electrode 3c is arranged in the coronary vein. In this case, when an electrical pulse is delivered between two electrodes, the electrical pulse is considered to pass through the route having the lowest electrical resistance among routes connecting between these electrodes. For example, when an electrical pulse is delivered between the electrodes 3b and 3c, the electrical pulse passes through the left ventricle side to stimulate a part of the left ventricular free wall E.

By so doing, the site to be stimulated by the defibrillation pulse can be more precisely selected.

In addition, in a case where the additional electrode 3c is arranged in the right atrium A, the superior vena cava C, or the inferior vena cava F, the additional electrode 3c may be placed at a certain position on either one of the two leads 4a and 4b which have been respectively arranged on the ventricular free walls D and E. By so doing, the third electrode 3c can be placed without adding the lead 4c, and the operation can be simplified as well as reducing the influence on the body of the patient.

In the abovementioned embodiment, the number of electrodes to be arranged in the heart B is not limited, and one more electrode may also be arranged in the heart B.

FIG. 7 illustrates an example where three electrodes are arranged on cardiac ventricular walls and one electrode 3d is arranged in the right ventricle. By so doing, the site of the heart B to be stimulated by electrical pulses can be further precisely selected.

The present invention has the following aspect.

The present invention provides an electrode layout method of a heart treatment apparatus comprising: inserting at least two lines of leads which are provided to the heart treatment apparatus and which have electrodes on their distal ends, into a vein communicated to the interior of the right atrium and extending along a cardiac wall; and placing the electrodes provided on the respective leads in the vein located at approximately opposite positions across the heart.

According to the present invention, since the leads can be transvenously inserted into the right atrium, there is no need of an open heart surgery and the invasion into the body of the patient due to the operation can be reduced. In addition, by supplying an electric stimulus to approximately opposite positions on the cardiac walls, the electric stimulus can be spread all over the cardiac walls. By so doing, the left and right cardiac walls can be effectively stimulated, and thereby the heart treatment efficiency can be improved.

In the abovementioned method, the electrodes are preferably arranged on the right ventricular free wall and the left ventricular free wall.

By so doing, the right ventricular free wall and the left ventricular free wall can be more effectively stimulated, and furthermore, the ventricular septum between the both ventricular free walls can also be effectively stimulated.

Moreover, in the abovementioned method, the electrode provided on either one of these leads may be arranged in the posterior vein of the left ventricle, the middle cardiac vein, or the great cardiac vein.

By so doing, the electrode can be readily arranged on the left ventricular free wall.

Furthermore, in the abovementioned method, the electrode provided on either one of these leads may also be arranged in an anterior cardiac vein or the small cardiac vein.

By so doing, the electrode can be readily arranged on the right ventricular free wall.

Those skilled in the art easily understand that the abovementioned veins include their branching veins.

In addition, in the abovementioned method, an electrode provided on an additional lead may also be placed at another position differing from the positions of the electrodes provided on these two leads, in the heart. In this case, the electrode provided on the additional lead is preferably arranged in the right ventricle, the right atrium, the superior vena cava, or the inferior vena cava.

By so doing, the site to be stimulated in the heart can be selected by outputting an electric stimulus between two electrodes among at least three electrodes arranged in the heart.

What is claimed is:

1. An electrode layout method of a heart treatment apparatus comprising: inserting at least two lines of leads which are provided to the heart treatment apparatus and which have electrodes on their distal ends, into a vein communicated to the interior of a right atrium and extending along a cardiac wall; and placing the electrodes provided on the respective leads in the vein located at approximately opposite positions across a heart.

2. An electrode layout method of a heart treatment apparatus according to claim 1, wherein said electrodes are arranged on a right ventricular free wall and a left ventricular free wall.

3. An electrode layout method of a heart treatment apparatus according to claim 1, wherein said electrode provided on either one of said leads is arranged in a posterior vein of the left ventricle.

4. An electrode layout method of a heart treatment apparatus according to claim 1, wherein said electrode provided on either one of said leads is arranged in a middle cardiac vein.

5. An electrode layout method of a heart treatment apparatus according to claim 1, wherein said electrode provided on either one of said leads is arranged in a great cardiac vein.

6. An electrode layout method of a heart treatment apparatus according to claim 1, wherein said electrode provided on either one of said leads is arranged in an anterior cardiac vein.

7. An electrode layout method of a heart treatment apparatus according to claim 1, wherein said electrode provided on either one of said leads is arranged in a small cardiac vein.

8. An electrode layout method of a heart treatment apparatus according to claim 1, wherein an electrode provided on an additional lead is placed at another position differing from the positions of said electrodes provided on said two leads, in the heart.

9. An electrode layout method of a heart treatment apparatus according to claim 8, wherein said electrode provided on the additional lead is arranged in a right ventricle.

10. An electrode layout method of a heart treatment apparatus according to claim 8, wherein said electrode provided on the additional lead is arranged in the right atrium.

11. An electrode layout method of a heart treatment apparatus according to claim 8, wherein said electrode provided on the additional lead is arranged in a superior vena cava.

12. An electrode layout method of a heart treatment apparatus according to claim 8, wherein said electrode provided on the additional lead is arranged in an inferior vena cava.
